(19) United States
(12) Patent Application Publication
(10) Pub. No.: US 2008/0145461 A1
(43) Pub. Date: Jun. 19, 2008

(54) Formulation and method of making a topical pain relief composition

(76) Inventor: Mark Gonzalez, Yorba Linda, CA (US)

Correspondence Address: MICHAEL A. SHIPPEY, PH. D., LAW OFFICES OF KARLA SHIPPEY, 4848 LAKEVIEW AVENUE, SUITE E, YORBA LINDA, CA, 92886, US

(21) Appl. No.: 11/639,611

(22) Filed: Dec. 14, 2006

(51) Int. Cl. A61K 36/534; A61P 25/00

(52) U.S. Cl. 424/747

(57) ABSTRACT

A topical pain relief composition for applying to the skin, comprising a mixture of stearic acid, emulsifying polymer, arnica oil, peppermint oil, and distilled water is disclosed.

Relation to Prior applications: None. This is an original application for patent. No federal funds were used in the development of the invention described herein.

### FIELD OF THE INVENTION

The invention relates to topical pain relief compositions for applying on the skin to alleviate subcutaneous discomfort associated with muscle strain or osteoarthritis and to promote tissue healing.

### BACKGROUND OF THE INVENTION

Compounds for topical pain relief have had widespread use. These compounds may provide pain relief in some circumstances when applied topically, however many products used to relieve pain only offer temporary relief and do not promote healing. Further, many topical pain relief compounds include camphor and/or capsaicin, which are known to cause skin irritation and are considered malodorous. Capsicum is an oleoresin obtained by extracting cayenne pepper with ether. It has been used for many years in compositions intended for pain relief, yet has undesirable skin irritants.

Patients having injured joints or damaged soft tissues generally do not benefit from these compounds, where they promote temporary pain relief but do little for tissue healing. Specifically, these compounds are ineffective topical carriers and do not transport tissue healing compounds past the skin to affected joint and soft tissue regions.

Peppermint oil has been used primarily as a flavoring agent, in aromatherapy, and as a cooling and soothing agent. However, it has not been explored as a pain reliever to the degree that camphor and capsicum have been used.

In other compounds using peppermint oil formulations as a pain reliever, see for example U.S. Pat. No. 7,029,711 (Farrell, S. L., Apr. 18, 2006) which teaches a transcutaneous composition containing inter alia glucosamine, peppermint and other flavoring oils, and a penetrating agent, dimethyl sulfoxide. Though these compounds having dimethyl sulfoxide as a topical carrier are known to reduce pain, skin irritation and unpleasant odors are common to the use thereof.

Other topical compositions for providing pain relief and anti-inflammatory action are known in the art, for example, U.S. Pat. No. 6,589,543 (McDaniels, L. M., Jul. 8, 2003) discloses a topical pain relief gel containing capsaicin, clove oil, ginger root powder, wintergreen and peppermint oils, and a base of petroleum jelly.

U.S. Pat. No. 6,555,125 (Campbell, P., Apr. 29, 2003) discloses a method for preparing pharmaceutical compositions for ulcer treatment. These contain antimicrobials, anti-inflammatory agents, and antihistamines, along with flavoring oils such as peppermint.

U.S. Pat. No. 5,560,910 (Crandall, W. T., Oct. 1, 1996) further discloses a topical anti-inflammatory composition containing bromelain, capsaicin and a penetrating agent selected from n-decylmethyl sulfoxide and lecithin organogel.

What is needed is a topical composition for application near a painful region to provide injured joint and soft tissues with elements that promote tissue healing, pain relief and muscle relaxation, and that is non-irritating to the skin, easily absorbed, fast acting and pleasant to smell.

### DETAILED DESCRIPTION

Detailed descriptions of the preferred embodiment are provided herein. It is to be understood, however, that the present invention may be embodied in various forms. Therefore, specific details disclosed herein are not to be interpreted as limiting, but rather as a basis for the claims and as representative basis for teaching one skilled in the art to employ the present invention in virtually any appropriately detailed system, structure or manner.

The present invention comprises novel topical compositions for application near painful regions to provide injured joint and soft tissues with elements that promote tissue healing, pain relief and muscle relaxation while being non-irritating to the skin, and are easily absorbed, fast acting and pleasant to smell. The advantages of the present invention emanate from the novel composition of the ingredients described below.

In one preferred embodiment, peppermint oil, having concentration between 3% and 30% by weight of the final composition is combined with cedarwood oil at between 0.8% and 15% by weight of the final composition, and thyme oil at a concentration of between 1-quarter and 1-half the level of cedarwood oil; wherein the combination is mixed at room temperature, between 60 and 75 degrees C., until the mixture combines into a single phase oil. A second mixture is then prepared as a carrier and emulsifyant for said first mixture by combining the ingredients polysorbate 60 having concentration between 0.8% and 15% by weight of the final composition; sorbitan monostearate having between 0.05% and 4.5% by weight of the final composition; Arnica flower tincture extract, having concentration between 0.8% and 15% by weight of the final composition; and Glyccyrrhizin at between 0.01% and 0.15% by weight of the final composition; in water; heating said second mixture to approximately 50 to 80 degrees C.; adding said first mixture to said second mixture to create a combined mixture; blending said combined mixture at high speed between 10,000 and 20,000 revolutions per minute until there is no separation, and the combined mixture is stiffened to a desired consistency. removing said combined mixture from heat.

This preferred embodiment produces a light viscosity, non-greasy ointment that is applied to the surface of the skin, i.e. topically, at an area of the body that is experiencing pain, fatigue, or irritation. The eyes and other sensitive areas of the body are not, preferably, allowed to come in contact with the ointment. Hands should be washed clean after use in the application of the ointment.

The ointment has a pleasant smell, primarily of peppermint. It can be applied frequently without noticeable side effects. However, of course, should side effects be observed, the application of the ointment should be discontinued.

Carbopol polymer can also be used in the formula to enhance the rate of emulsification.

In house observations have detected no separation or degradation of the emulsified product of the current invention for over two years of standing in a jar.

It is believed that the active ingredients of the preferred embodiment are these: peppermint oil, with active range 3 to 30% by weight of the ointment; Arnica extract, at 0.8 to 15% by weight of the ointment; and Glycyrrhizin at 0.01% to 0.5% weight of the ointment. These ingredients combine to form a potent pain relieving formulation. Glycyrrhizin is obtainable as an extract from licorice.

Additionally of importance in the preferred embodiment are sorbitan monostearate, and polysorbate 60. These are the agents that combine with water to form the base of the ointment. They also act as emulsifying agents.

I claim:

1. A topical pain relief composition for applying to the skin, comprising effective amounts of a mixture of: peppermint oil; arnica extract; Glycyrrhizin; distilled water.

2. The topical pain relief composition of claim 1, comprising the following components, prepared in any quantity by maintaining equivalent ratios: peppermint oil 5.0% arnica oil 1.00% Glycyrrhizin 0.01% distilled water 64.0% wherein the amounts of any of said components may vary by plus or minus up to 20%.

3. The topical pain relief composition of claim 1, wherein the effective range of said peppermint oil is between 3% and 30% by weight of said composition.

4. The topical pain relief composition of claim 1, wherein the effective range of said arnica extract is between 0.8% and 12% by weight of said composition.

5. The topical pain relief composition of claim 1, wherein the effective range of said Glycyrrhizin is between 0.01% and 0.50% by weight of said composition.

6. The topical pain relief composition of claim 1, further comprising carbopol polymer at between 1% and 10% by weight of said composition.

7. A method for the production of the topical pain relief composition of claim 1, said method comprising: Combining Peppermint oil, having concentration between 3% and 30% by weight of the final composition with cedarwood oil at between 0.8% and 15% by weight of the final composition, and thyme oil at a concentration of between 1-quarter and 1-half the level of cedarwood oil; whereupon the combination is mixed at room temperature, between 60 and 75 degrees C., until the mixture combines into a single phase oil;
Preparing a second mixture as a carrier and emulsifyant for said first mixture by combining the ingredients polysorbate 60 having concentration between 0.8% and 15% by weight of the final composition; sorbitan monostearate having between 0.05% and 4.5% by weight of the final composition; Arnica flower tincture extract, having concentration between 0.8% and 15% by weight of the final composition; and Glyccyrrhizin at between 0.01% and 0.15% by weight of the final composition; in water; heating said second mixture to approximately 50 to 80 degrees C.; adding said first mixture to said second mixture to create a combined mixture; blending said combined mixture at high speed between 10,000 and 20,000 revolutions per minute until there is no separation, and the combined mixture is stiffened to a desired consistency, and,
then removing said combined mixture from heat.
